(19) United States
(12) Patent Application Publication
(10) Pub. No.: US 2006/0041313 A1
(43) Pub. Date: Feb. 23, 2006

(54) Intervertebral disc system

(75) Inventors: Randall Allard, Germantown, TN (US); Elliott Marshall, Seattle, WA (US); Leonard Tokish JR., Issaquah, WA (US); Alex Kunzler, La Quinta, CA (US); David Yager, Carnation, WA (US); Tom Francis, Cordova, TN (US); Kenneth Misser, Bellevue, WA (US); Greg Marik, Germantown, TN (US); Kevin Foley, Germantown, TN (US); David Rosler, Seattle, WA (US); Lukas Eisermann, Memphis, TN (US); Anthony Finazzo, Lake Forest Park, WA (US); Richard J. Broman, Monroe, WA (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/131,758

(22) Filed: May 18, 2005

Related U.S. Application Data
(63) Continuation of application No. 10/922,094, filed on Aug. 19, 2004, now abandoned.

(51) Int. Cl. A61F 2/44

(52) U.S. Cl. 623/17.15; 623/17.16; 623/17.11

(57) ABSTRACT

An vertebral implant is interposed between two vertebral endplates and comprises a first endplate assembly having a first restraint mechanism extending from a first exterior surface for engaging a first vertebral endplate The implant further comprises a second endplate assembly having a second restraint mechanism extending from a second exterior surface for engaging a second vertebral endplate and a central body articulable between the first and second endplate assemblies. The first restraint mechanism has a shape that matches a contour in the first vertebral endplate.

### CROSS-REFERENCE

This application is a continuation of U.S. patent application No. 10/922,094 filed Aug. 19, 2004, and entitled, “Intervertebral Disc System,” which is hereby incorporated by reference in its entirety.

### BACKGROUND

During the past thirty years, technical advances in the design of large joint reconstructive devices has revolutionized the treatment of degenerative joint disease, moving the standard of care from arthrodesis to arthroplasty. Progress in the treatment of vertebral disc disease, however, has come at a slower pace. Currently, the standard treatment for disc disease remains discectomy followed by vertebral fusion. While this approach may alleviate a patient's present symptoms, accelerated degeneration of adjacent discs is a frequent consequence of the increased motion and forces induced by fusion. Thus, reconstructing the degenerated intervertebral disc with a functional disc prosthesis to provide motion and to reduce deterioration of the adjacent discs may be a more desirable treatment option for many patients.

### SUMMARY

In one embodiment, a vertebral implant is interposed between two vertebral endplates and comprises a first endplate assembly having a first restraint mechanism extending from a first exterior surface for engaging a first vertebral endplate The implant further comprises a second endplate assembly having a second restraint mechanism extending from a second exterior surface for engaging a second vertebral endplate and a central body articulable between the first and second endplate assemblies. The first restraint mechanism has a shape that matches a precision contour in the first vertebral endplate.

In another embodiment, a vertebral implant comprises a central body articulable between first and second endplate assemblies and a method of implant the vertebral implant between two vertebral endplates comprises positioning a rotable burr between a first vertebral endplate and a second vertebral endplate. The rotable burr is moved in a transverse direction, and bone is removed from a first vertebral endplate to form a first contour. The implant is inserted between the first and second endplate assemblies, and the first endplate assembly is positioned in contact with the first contour. The shape of the first endplate assembly matches the shape of the first contour.

### BRIEF DESCRIPTION OF THE DRAWINGS

FIG. 1 is a side view of vertebral column having a destroyed disc.

FIG. 2 is a side view of a vertebral column having a vertebral prosthesis.

FIG. 3 is a perspective view of an prosthesis according to a first embodiment of the present invention.

FIG. 4 is a cross-sectional view of the prosthesis according to the first embodiment of the present invention.

FIG. 5 is a cross-sectional view of a prosthesis according to a second embodiment of the present invention.

FIG. 6 is a perspective view of the prosthesis according to the second embodiment of the present invention.

FIG. 7 is a cross-sectional view of a prosthesis according to a third embodiment of the present invention.

FIG. 8 is a perspective view of the prosthesis according to the third embodiment of the present invention.

FIG. 9 is a cross-sectional view of a prosthesis according to a fourth embodiment of the present invention.

FIG. 10 is a perspective view of the prosthesis according to the fourth embodiment of the present invention.

FIG. 11 is a cross-sectional view of a prosthesis according to a fifth embodiment of the present invention.

FIG. 12A is a perspective view of the prosthesis according to the fifth embodiment of the present invention.

FIG. 12B is a perspective view of the prosthesis according to the fifth embodiment of the present invention.

FIG. 12C is a perspective view of the prosthesis according to a sixth embodiment of the present invention.

FIG. 12D is a perspective view of the prosthesis according to a seventh embodiment of the present invention.

FIG. 13 is a perspective view of a tool used for prosthesis implantation.

FIG. 14 is a perspective view of a fixture for inserting an intervertebral disc prosthesis.

FIGS. 15-18 are views of a tool for milling bone.

FIGS. 19-21 are views of tools for controlling the milling of bone.

FIG. 22 is a perspective view of a tool for inserting a prosthesis.

### DETAILED DESCRIPTION

The present invention relates generally to vertebral reconstructive devices, and more particularly, to a functional intervertebral disc prosthesis. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

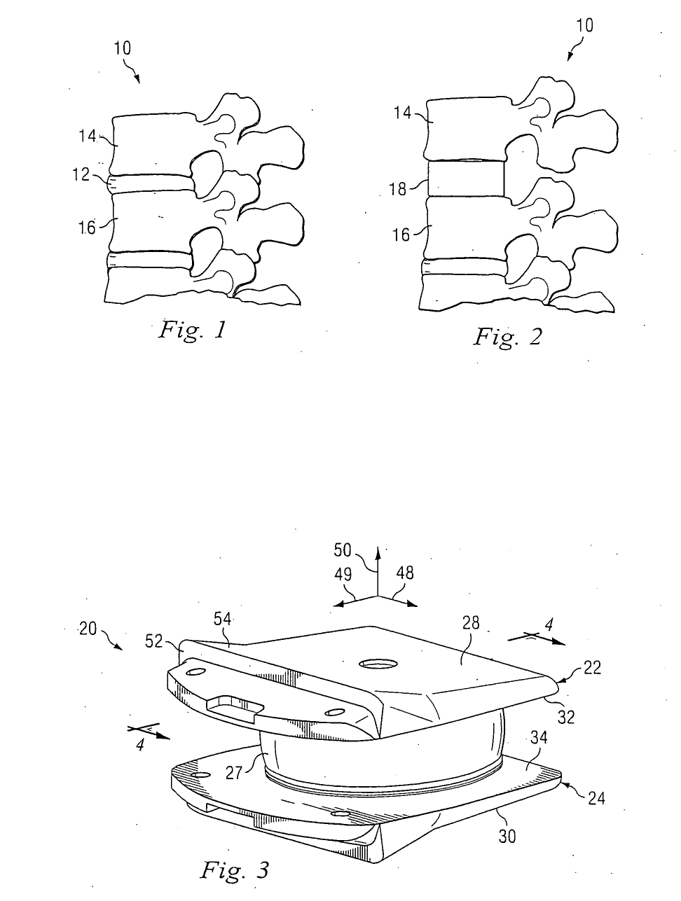

Referring first to FIG. 1, the reference numeral 10 refers to a vertebral column with a damaged intervertebral disc 12 extending between two intact vertebrae 14 and 16. In a typical surgical discectomy, the damaged disc 12 is removed creating a void between the two intact vertebra 14 and 16. This procedure may be performed using an anterior, anterolateral, lateral, or other approach known to one skilled in the art. Referring now to FIG. 2, a prosthesis 18 may be provided to fill the void between the vertebrae 14 and 16. In broad aspect, the size and shape of the prosthesis 18 are substantially variable, and this variation will depend upon the joint geometry. Moreover, a prosthesis 18 of a particular shape can be produced in a range of sizes, so that a surgeon can select the appropriate size prior to or during surgery, depending upon his assessment of the joint geometry of the patient, typically made by assessing the joint using CT, MRI, fluoroscopy, or other imaging techniques. In the embodiments to be described, the prosthesis 18 may articulable, restoring a range of motion to the affected spinal joint. Where articulation may not be desirable, however, the prosthesis 18 may be adapted to permit fusion. The prosthesis 18 may work in cooperation with existing facets, annulus fibrosus, ligamentous and muscular soft tissues to allow kinematics typical of various areas of the spine, including the lumbar region.

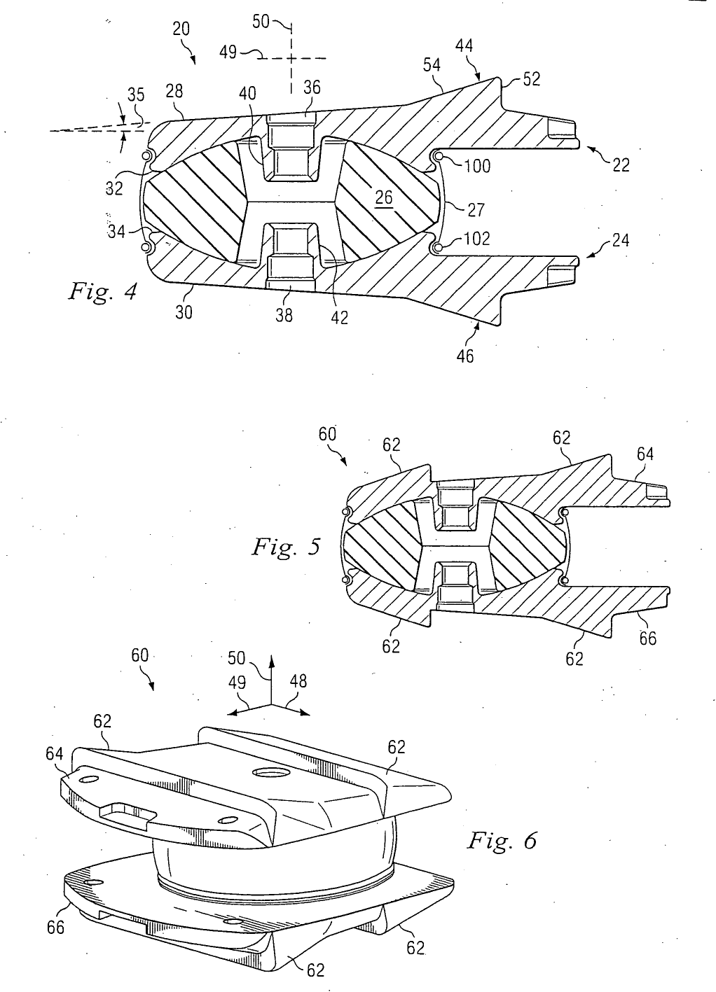

Referring now to FIGS. 3-4, an intervertebral disc prosthesis 20 may be used as the prosthesis 18 of FIG. 2. The intervertebral disc prosthesis 20, according to an embodiment of the present invention, includes endplate assemblies 22, 24 between which a central body 26 may extend. A flexible sheath 27 may extend between the endplate assemblies 22, 24, encapsulating the central body 26.

The endplate assemblies 22, 24 may include exterior surfaces 28, 30 respectively and interior surfaces 32, 34 respectively. The exterior surfaces 28, 30 may be relatively flat as shown in FIG. 3-4, but in other embodiments, the exterior surface may have a curved or domed shape. The exterior surfaces 28, 30 may match precision milled vertebral endplates as will be described below. At least a portion of the interior surfaces 32, 34 may be smooth and of a shape, such as concave or convex, that complements and articulates with the shape of at least a portion of the central body 26. The articulating portion of the interior surfaces 32, 34 may be offset such that when implanted, the central body 26 may be placed in a posterior position to achieve more natural spinal kinematics. This smoothness and correspondence in shape may provide unconstrained movement of the endplate assemblies 22, 24 relative to the central body 26, provided that this movement occurs within the allowable range of motion.

The structural features of the shapes of the interior surface 32, 34 and the central body 26 that interact to limit the movement to this allowable range may vary to some extent, based on the joint in which the implant will be used. The endplate assemblies 22, 24 may be identical, to simplify manufacturing, or alternatively, may be of different design (shape, size, and/or materials) to achieve different mechanical results. For example, differing endplate assemblies may be used to more closely tailor the implant to a patient's anatomy, or to shift the center of rotation in the cephalad or caudal direction.

As shown in the embodiment of FIG. 4, the endplate assemblies 22, 24 and the central body 26 can contain complementary structures that will function as an expulsion stop so that the central body 26 may not be expelled from between the endplate assemblies 22, 24 when the endplate assemblies are at maximum range of motion in flexion/extension. Such structures may also be used to partially constrain the central body 26 within an allowable range of motion. Examples of such structures, as shown in FIG. 4, may include posts 36, 38 extending from the interior surfaces 32, 34 respectively. Corresponding recesses 40, 42 in the central body 26 may receive the posts 36, 38, respectively. The recesses 40, 42 may be sized sufficiently large that relative motion between the endplate assemblies and central body is unconstrained within an allowable range of motion, but that will nevertheless cause the posts 36, 38 to arrest the central body before it is expelled from the implant under extreme compression, flexion, extension, or translation.

The endplate assemblies 22, 24 can be made of any rigid, biocompatible material, including a biocompatible metal, such as stainless steel, cobalt chromium, ceramics, such as those including Al₂O₃ or Zr₂O₃, or a titanium alloy such as ASTM F-136 titanium alloy. The exterior surfaces 28, 30 may be rough in order to restrict motion of the endplate assemblies relative to the bone surfaces that are in contact with the plates. A rough or porous coating (not shown), which may be formed from nonspherical sintered beads, can provide very high friction between the exterior surfaces 28, 30 of the endplate assemblies and the adjacent bone, as well as providing a suitable interaction with the cancellous bone of the joint, increasing the chances of bony ingrowth. One example of a suitable nonspherical sintered bead coating is that made of pure titanium, such as ASTM F-67. The coating may be formed by vacuum sintering. Other suitable treatments may include hydroxyapatite, osteogenic peptide coating, growth factor coating, rh-BMP coating, and grit blasting.

As also shown in FIGS. 3-4, the endplate assemblies 22, 24 may include structures that function as restraint mechanisms to aid in securing the assemblies to the adjacent bone. For example, tabs 44, 46 may project from the exterior surfaces 28, 30 respectively. The tabs 44, 46 may be formed in any of a variety of configurations including, as shown in the embodiment of FIG. 4, a single angled projection which may extend transversely, along an axis 48, over at least a portion of the exterior surface 28. The tabs 44, 46 may be longer along the transverse axis 48 than along an axis 49 in the anterior-posterior direction and may project away from the exterior surfaces 28, 30 in an axial direction 50. The tab 44 may have a face 52 extending at a perpendicular or oblique angle from the exterior surface 28. The tab 44 may also have face 54 extending between the face 52 and the exterior surface 28. The tab 46 may be similarly or identically configured and therefore will not be described in detail.

Referring again to FIGS. 3-4, the endplate assemblies 22, 24 may be angled to achieve desirable lordotic or kyphotic loading. An angle 35 may be formed between exterior surface 28 of the endplate assembly 22 and the anterior-posterior axis 50. In some embodiments, the angle 35 may be between 8 and 20 degrees. The endplate assembly 24 may be similarly configured.

Other embodiments, as shown in FIGS. 5-10 may include projections which can vary in quantity and in shape. The projections may be formed to match precision-milled grooves in adjacent bone structures, such the endplates of vertebrae 14, 16. As shown in the embodiment of FIGS. 5-6, a prosthesis 60, which may be used as the prosthesis 18 of FIG. 2, may include a plurality of projections 62 extending from exterior surfaces 64, 66. Each projection 62 may be configured similarly to tab 44 as described above.

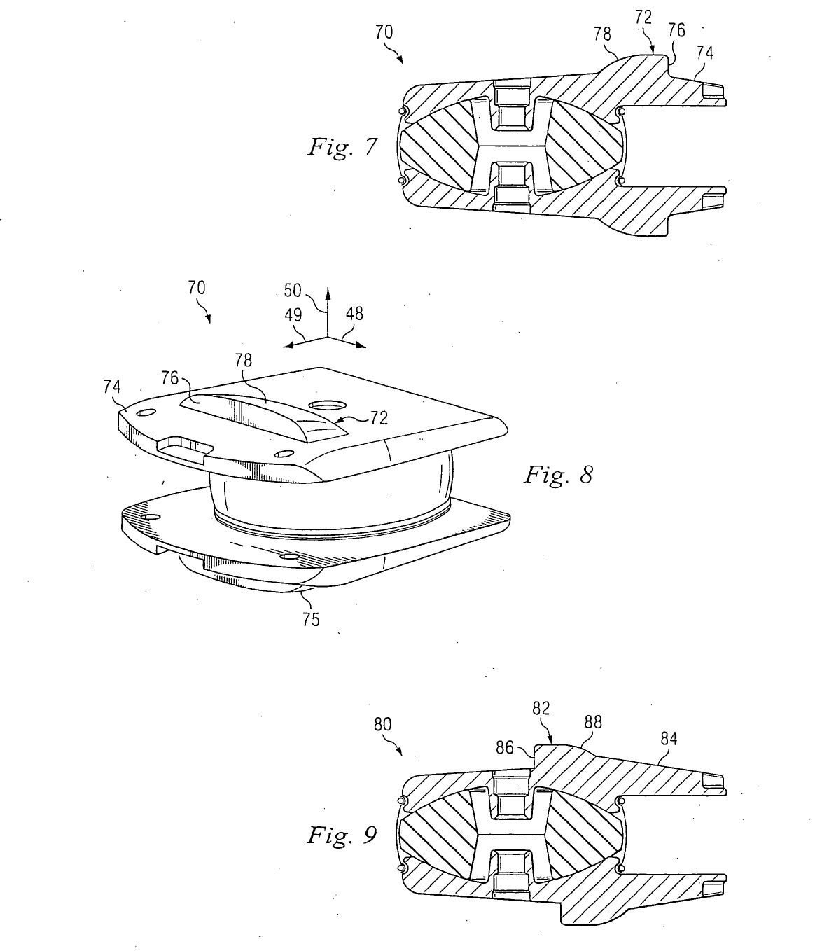

In another embodiment, as shown in FIGS. 7-8, a prosthesis 70, which may be used as the prosthesis 18 of FIG. 2, may include a tab 72 extending from an exterior surface 74. Each tab 72 may include a face 76 extending at a perpendicular or oblique angle from the exterior surface 74. The tab 72 may also have a face 78 extending between the face 76 and the exterior surface 74. The face 78 may be curved and/or D-shaped. A corresponding tab may be located on the exterior surface of the opposite endplate assembly. The face 78 may extend from the face 76 in a posterior direction along the axis 49 to prevent slippage of the implanted prosthesis 70 in the anterior direction.

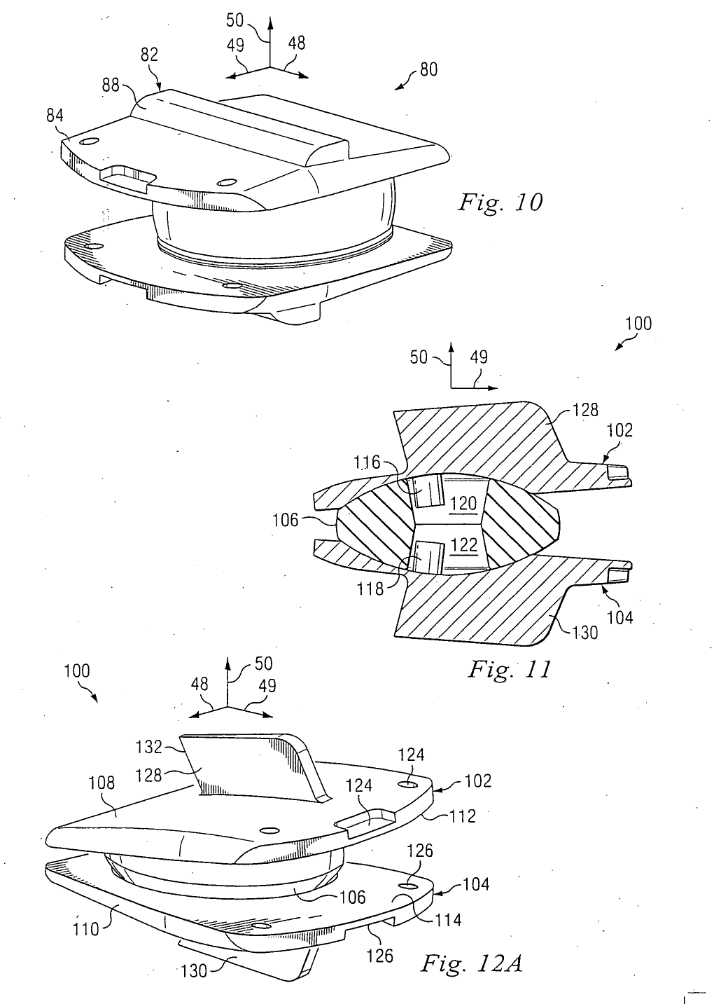

In another embodiment, as shown in FIGS. 9-10, a prosthesis 80, which may be used as the prosthesis 18 of FIG. 2, may include a tab 82 extending from an exterior surface 84. Each tab 82 may include a face 86 extending at a perpendicular or oblique angle from the exterior surface 84. The tab 82 may also have a face 88 extending between the face 86 and the exterior surface 84. The face 88 may be curved and/or D-shaped. A corresponding tab may be located on the exterior surface of the opposite endplate assembly. The face 88 may extend from the face 86 in an anterior direction along the axis 49 to prevent slippage of the implanted prosthesis 80 in the posterior direction.

The central body 26 may vary somewhat in shape, size, composition, and physical properties, depending upon the particular joint for which the implant is intended. The shape of the central body 26 may complement that of the inner surface of the endplate assembly to allow for a range of translational, flexural, extensional, and rotational motion, and lateral bending appropriate to the particular joint being replaced.

A desirable degree of elasticity or dampening may be provided by the thickness and physical properties of the central body 26. Accordingly, an elastomeric material may be used for the central body. Although flexible, the central body 26 may be sufficiently stiff to effectively cooperate with the endplate assemblies 22, 24 to limit motion beyond the allowable range. The surface of the central body 26 may also be sufficiently durable to provide acceptable wear characteristics. In one embodiment, this combination of properties may be achieved with a central body 26 having surface regions that are harder than the material of the central body closer to its core. The central body 26 may, therefore, comprise a biocompatible elastomeric material having a hardened surface. Polyurethane-containing elastomeric copolymers, such as polycarbonate-polyurethane elastomeric copolymers and polyether-polyurethane elastomeric copolymers, generally having durometer ranging from about 80A to about 65D (based upon raw, unmolded resin) may be suitable for vertebral applications.

If desired, these materials may be coated or impregnated with substances to increase their hardness or lubricity, or both. Coating may be done by any suitable technique, such as dip coating, and the coating solution may include one or more polymers, including those described below for the central body. The coating polymer may be the same as or different from the polymer used to form the central body 26, and may have a different hardness from that used in the central body. Coating thickness may be greater than approximately 1 mil, with some embodiments having coating thicknesses of about 2 mil to about 5 mil. Examples of suitable materials include ultra-high molecular weight polyethylene (UHMWPE), polyurethanes, such as polycarbonates and polyethers, such as Chronothane P 75A or P 55D (P-eth-PU aromatic, CT Biomaterials); Chronoflex C 55D, C 65D, C 80A, or C 93A (PC-PU aromatic, CT Biomaterials); Elast-Eon II 80A (Si-PU aromatic, Elastomedic); Bionate 55D/S or 80A-80A/S (PC-PU aromatic with S-SME, PTG); CarboSil-10 90A (PC-Si-PU aromatic, PTG); Tecothane TT-1055D or TT-1065D (P-eth-PU aromatic, Thermedics); Tecoflex EG-93A (P-eth-PU aliphatic, Thermedics); and Carbothane PC 3585A or PC 3555D (PC-PU aliphatic, Thermedics).

As shown in FIG. 4, the sheath 27 may be a tubular structure, and is made from a flexible material. The material used to make the sheath may be biocompatible and elastic, such as a segmented polyurethane, having a thickness ranging from about 5 to about 30 mils, more particularly about 10-11 mils. Examples of suitable materials include BIOSPAN-S (aromatic polyetherurethaneurea with surface modified end groups, Polymer Technology Group), CHRONOFLEX AR/LT (aromatic polycarbonate polyurethane with low-tack properties, CardioTech International), CHRONOTHANE B (aromatic polyether polyurethane, CardioTech International), CARBOTHANE PC (aliphatic polycarbonate polyurethane, Thermedics).

Referring still to FIGS. 3-4, the various geometric features of the main components of this embodiment may cooperate to join the components into a unitary structure. In general, the ends of the sheath 27 are attached to the endplate assemblies 22, 24, and the central body 26 is encapsulated between the endplate assemblies and the sheath. More specifically, referring to FIG. 4, retaining rings 100, 102 may then placed over the edges of the sheath 27 and into a set of circumferential grooves 94, 96, thereby holding the flexible sheath 27 in place and attaching it to the endplate assemblies. Any suitable biocompatible material can be used for the retaining rings, including titanium or titanium alloys, such as nitinol. The retaining rings may be fixed in place by, for example, welding the areas of overlap between the ends of the retaining rings. After the sheath 27 is attached, a liquid lubricant (not shown), such as saline, may be introduced to at least partially fill the space around the central body 26.

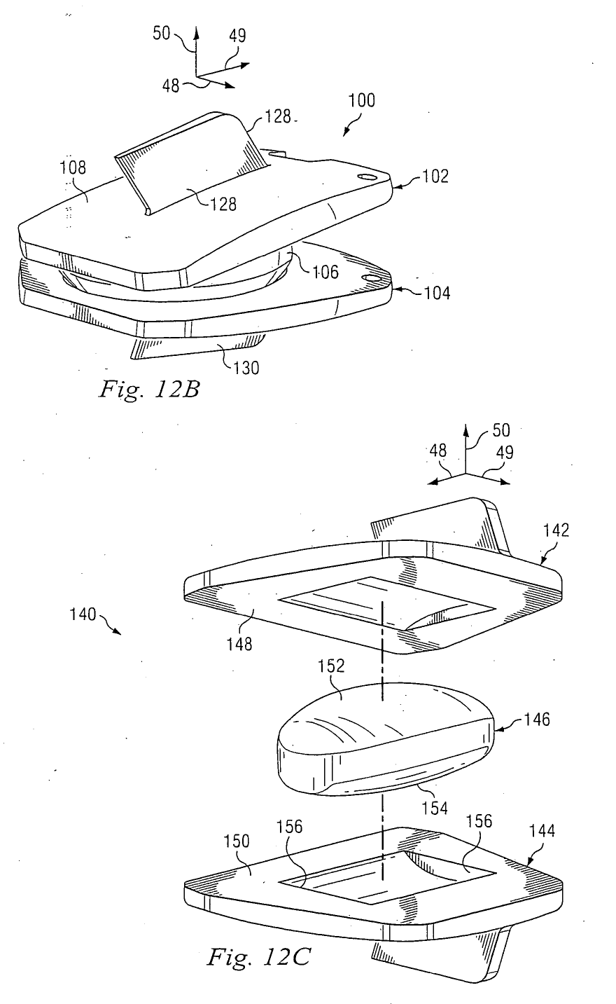

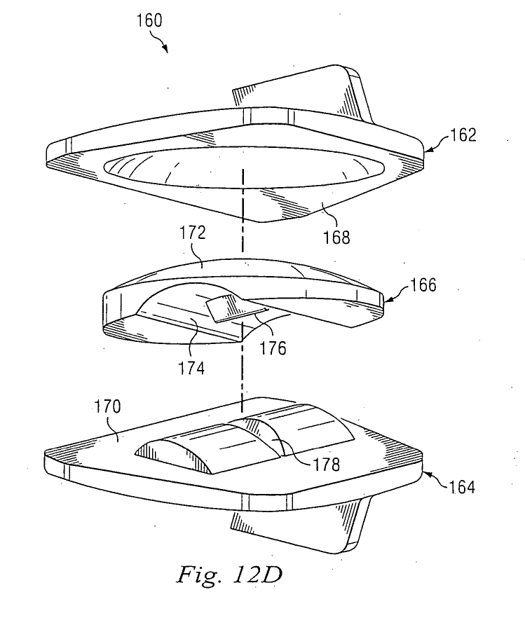

Referring now to FIGS. 11, 12A, 12B, an intervertebral disc prosthesis 100 may be used as the prosthesis 18 of FIG. 2. The intervertebral disc prosthesis 100, according to an embodiment of the present invention, includes endplate assemblies 102, 104 between which a central body 106 may extend.

The endplate assemblies 102, 104 may include exterior surfaces 108, 110 respectively and interior surfaces 112, 114 respectively. The exterior surfaces 108, 110 may be relatively flat, tapered, curved, domed, or any other shape conducive to implantation, vertebral endplate mating, or revision. The exterior surfaces 108, 110 may match precision milled vertebral endplates. At least a portion of the interior surfaces 112, 114 may be smooth and of a shape, such as concave, that complements and articulates with the shape of at least a portion of the central body 106. The articulating portion of the interior surfaces 112, 114 may be offset such that when implanted, the central body 106 may be placed in a posterior position to achieve more natural spinal kinematics. In other embodiments, the central body 106 may be placed in a relatively anterior position. The smoothness and correspondence of the shape may provide unconstrained movement of the endplate assemblies 102, 104 relative to the central body 106, provided that this movement occurs within the allowable range of motion.

The structural features of the shapes of the interior surface 112,114 and the central body 106 that interact to limit the movement to this allowable range may vary to some extent, based on the joint in which the implant will be used. The endplate assemblies 102, 104 may be identical, to simplify manufacturing, or alternatively, may be of different design (shape, size, and/or materials) to achieve different mechanical results. For example, differing endplate assemblies may be used to more closely tailor the implant to a patient's anatomy, or to shift the center of rotation in the cephalad or caudal direction.

The exterior surfaces 108, 110 may include tool engagement elements 124, 126, such as recesses, protrusions, apertures or other structures, which may be accessed by an insertion, positioning, or revision tool to engage the prosthesis 100. The exterior surfaces 108, 110 may be tapered toward the intended direction of implantation to assist with implantation. In this embodiment, the exterior surfaces 108, 110 taper away from the direction of the engagement elements 124, 126. In some embodiments (as shown more clearly in FIG. 12C) the endplate assemblies 102, 104 may be trapezoidal shape to allow balancing between cancellous and cortical bone area when the prosthesis 100 is placed in use.

As shown in the embodiment of FIG. 11, the endplate assemblies 102, 104 and the central body 106 can contain complementary structures that will function as an expulsion stop so that the central body 106 may not be expelled from between the endplate assemblies 102, 104 when the endplate assemblies are at maximum range of motion in flexion/extension. Such structures may also be used to partially constrain the central body 106 within an allowable range of motion. Examples of such structures, as shown in FIG. 11, may include posts 116, 118 extending from the interior surfaces 112, 114 respectively. Corresponding recesses 120, 122 in the central body 106 may receive the posts 116, 118, respectively. The recesses 120, 122 may be sized sufficiently large that relative motion between the endplate assemblies and central body is unconstrained within an allowable range of motion, but that will nevertheless cause the posts 116, 118 to arrest the central body before it is expelled from the implant under extreme compression, flexion, extension, or translation.

The endplate assemblies 102, 104 may be made of any rigid, biocompatible material, including a biocompatible metal, such as stainless steel, cobalt chromium, ceramics, such as those including Al₂O₃ or Zr₂O₃, or a titanium alloy such as ASTM F-136 titanium alloy. The exterior surfaces 108, 110 may be rough in order to restrict motion of the endplate assemblies relative to the bone surfaces that are in contact with the plates. A rough or porous coating (not shown), which may be formed from nonspherical sintered beads, can provide very high friction between the exterior surfaces 108, 110 of the endplate assemblies and the adjacent bone, as well as providing a suitable interaction with the cancellous bone of the joint, increasing the chances of bony ingrowth. One example of a suitable nonspherical sintered bead coating is that made of pure titanium, such as ASTM F-67. The coating may be formed by vacuum sintering. Other suitable treatments may include hydroxyapatite, osteogenic peptide coating, growth factor coating, rh-BMP coating, and grit blasting. The central body 106 may comprise any of the materials described above for central body 26.

As also shown in FIGS. 11, 12A, 12B, the endplate assemblies 102, 104 may include structures that function as restraint mechanisms to aid in seating the prosthesis 100, securing the endplate assemblies 102, 104 to the adjacent bone, or revising the prosthesis 100. For example, tabs 128, 130 may project from the exterior surfaces 108, 110 respectively. In some embodiments, only one endplate assembly may be provided with a tab. The tab 128 may be keel-shaped and extend along the axis 49, in an anterior-posterior direction when inserted. The length of the tab 128 long the axis 49 may be longer than the width of the tab 128 along the transverse axis 48. The tab 128 may have a tapered end 132 to aid with the insertion of the prosthesis 100. The tab 128 may also have a forward rake to permit self-cutting of the vertebral endplate and to enhance seating. Forward placement of the tab 128 (towards the anterior direction in this embodiment) may minimize the force required to install the prosthesis 100, create a greater safety margin to the posterior aspect, minimize the machining of the vertebral endplates, and provide a visual cue for seating. The tab 128 may be wedge-shaped or tapered from a distal edge toward the exterior surface 108 to enhance the purchase of the seated prosthesis 100. In some embodiments, for example where revision may be anticipated, the tab 128 may be polished or otherwise prepared to resist bone in-growth. In some embodiments, the tab 128 may have apertures (not shown) or other surface coatings to permit bone in-growth. The tab 130 may be similarly or identically configured and therefore will not be described in detail.

Referring now to FIG. 12C, an intervertebral disc prosthesis 140 may be used as the prosthesis 18 of FIG. 2. This prosthesis 140 may form a constrained joint such as has been described in U.S. patent applications [Attorney Reference Number PC 1005.00 and PC1006.00] entitled “Constrained Artificial Spinal Disc” and “Constrained Artificial Implant for Orthopaedic Applications,” respectively, which are hereby incorporated by reference. The intervertebral disc prosthesis 140, according to an embodiment of the present invention, includes endplate assemblies 142, 144 between which a central body 146 may extend. The endplates 142, 144 may comprise interior surfaces 148, 150, respectively. The endplate assemblies 142, 144 may be configured the same as or similar to endplate assemblies 102, 104 of FIGS. 11, 12a, 12b (with certain exceptions including those noted below) and therefore will not be described in extensive detail.

Center body 146 may have a convex cephalad surface 152 shaped to articulate with concave portion of interior surface 148 and a convex caudal surface 154 shaped to articulate with a concave portion of interior surface 150. In this embodiment, the surface 152 has a shallower convexity than the surface 154 which may promote a tendency for the prosthesis 140 to self-align along the cephalad-caudal axis 50 when the prosthesis 140 is subjected to loading. In this embodiment, lateral motion between the center body 146 and the endplate assembly 144 may be limited by stops 156. The central body 146 may be formed from any of the materials described above for central body 26.

Referring now to FIG. 12D, an intervertebral disc prosthesis 160 may be used as the prosthesis 18 of FIG. 2. This prosthesis 160 may also form a constrained joint such as has been described in U.S. patent applications [Attorney Reference Number PC1005.00 and PC1006.00] entitled “Constrained Artificial Spinal Disc” and “Constrained Artificial Implant for Orthopaedic Applications,” respectively. The intervertebral disc prosthesis 160, according to an embodiment of the present invention, includes endplate assemblies 162, 164 between which a central body 166 may extend. The endplates 162, 164 may comprise interior surfaces 168, 170, respectively. The endplate assemblies 162, 164 may be configured the same as or similar to endplate assemblies 102, 104 of FIGS. 11, 12a, 12b (with certain exceptions including those noted below) and therefore will not be described in extensive detail.

Center body 166 may have a convex cephalad surface 172 shaped to articulate with concave portion of interior surface 148 and a concave caudal surface 174 shaped to articulate with a convex portion of interior surface 170. In this embodiment, the surface 172 has a shallower curvature than the surface 174 which may promote a tendency for the prosthesis 160 to self-align along the cephalad-caudal axis 50 when the prosthesis 160 is subjected to loading. In this embodiment, lateral motion between the center body 166 and the endplate assembly 164 may be limited by a stop projection 176 on the caudal surface 174 of the central body 166 matingly engaged with a stop recess 178 on the convex portion of the interior surface 170. The central body 166 may be formed from any of the materials described above for central body 26.

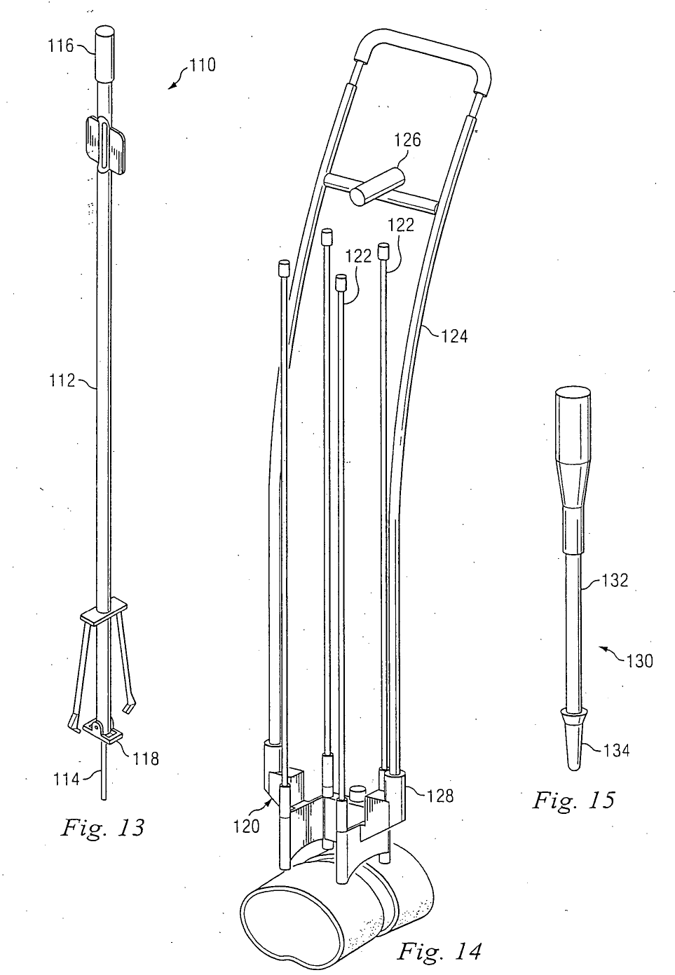

Referring now to FIGS. 13-22, a series of implantation devices may be used to prepare the space between the vertebrae 14, 16 to receive the prosthesis 18. The space between the vertebrae 14, 16 may be distracted and a depth measurement instrument 210, as shown in FIG. 13, may be inserted between the vertebrae 14, 16 to measure an average height. The depth measurement instrument 210 may comprise a shaft 212 extending between a probe 214 and a handle 216. The probe 214 may comprise a foot element 218. The probe 214 may be inserted between the distracted vertebrae 14, 16 with the foot element 218 positioned on the anterior surface of the vertebrae. The foot element 218 may pivot to provide an average depth measurement.

Referring now to FIG. 14, a milling fixture 220 may be attached to the vertebral bodies 14, 16 with a plurality of fixation devices 222, such as flexible screws which may be bent to permit access and a line of sight into the area between the vertebral bodies. A handle 224 may extend from the milling fixture 220 and may comprise a locking element 226 to lock the handle 224 to a rigid reference point. When not in use, the handle 224 may be disconnected from the milling fixture 220 by activating a quick connect device 228.

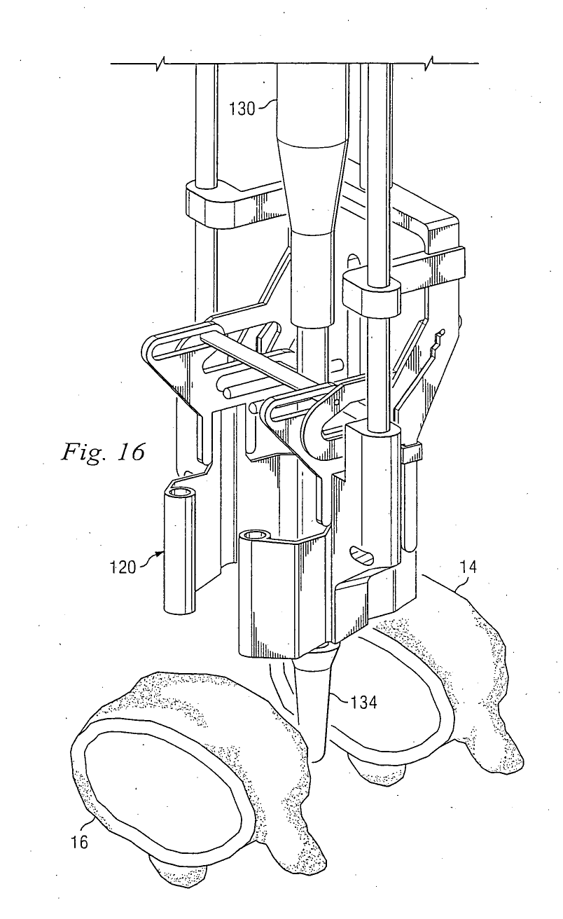

Referring now to FIG. 15-16, a milling device 230 may comprise a handle 232 and one or more milling elements 234 which may be a rotary cutting tool such as an axial profile burr. A burr may be provided in any of a variety of shapes including a bulbous or tapered shape. As shown in FIG. 16, the milling element 234 may be positioned within the distracted space between the vertebrae 24, 26 and aligned using the milling fixture 220 and handle 224.

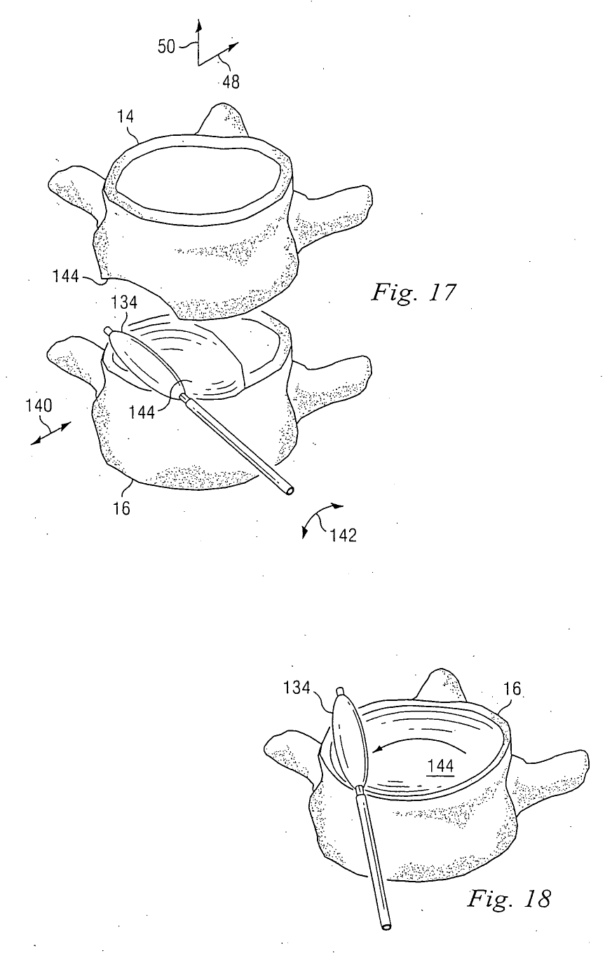

As shown in FIGS. 17-18, the milling element 234 may rotate while being moved in the transverse direction 48 with a linear motion 240 or an arc or swing motion 242 to create precision milled contours 244 in vertebrae 14, 16. The contours 244 may correspond to the shape of the prosthesis 18, including the shape of the prosthesis 18 exterior surfaces and projections.

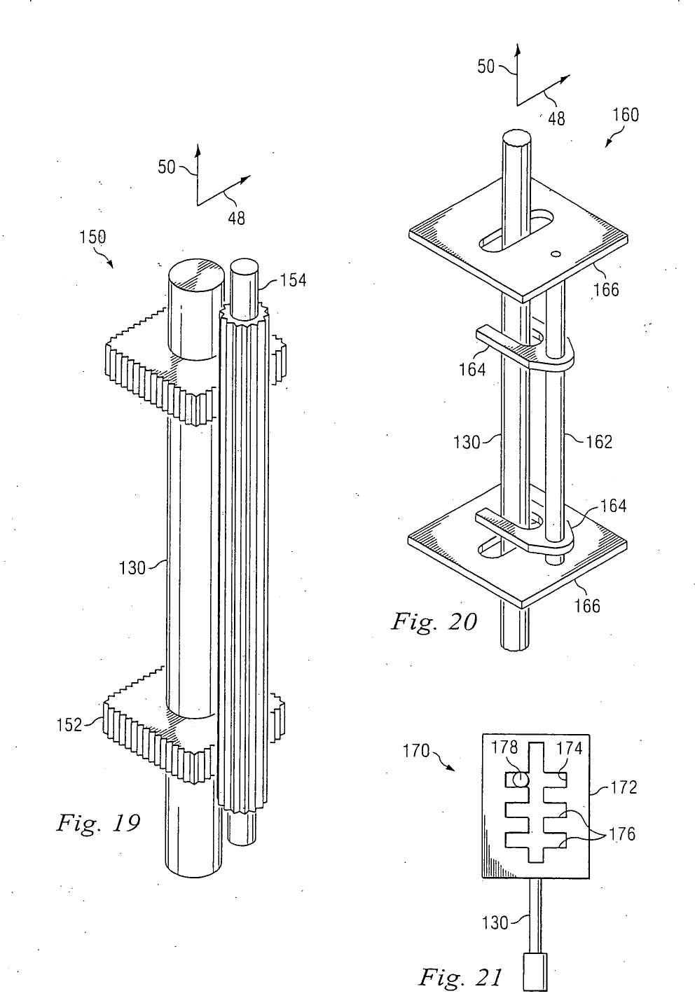

Referring now to FIG. 19-20, the motion 240 or 242 may be controlled by any of a variety of mechanisms connected to the milling fixture 220 (FIG. 14). As shown in FIG. 19, a motion control device 250 may include a rack 252 connected to the milling device 230 and a pinion gear 254 that may engage the rack 252. As the pinion gear 254 rotates in place, the rack 252 may translate in the transverse direction 48. As shown in FIG. 20, a motion control device 260 may be a pivoting yoke system including a rod 262 rigidly connected to one or more yoke devices 264. The yoke devices 264 may moveably engage the milling device 230. As the rod 252 is pivoted in place, the milling device 230 may translate in the transverse direction 228. One or more slotted plates 266 may be provided to guide the motion of the milling device 230.

Referring now to FIG. 21, milling of the vertebrae 14, 16 may also be controlled by a ratchet assembly 270 which may include a ratchet housing 272 for housing a ratchet mechanism 274. The ratchet mechanism 274 may include a plurality of ratchet positions 276 and an attachment device 278, such as a pin, for engaging the milling device 230 with the ratchet mechanism 274. The plurality of ratchet positions 276 allow the milling device 230 to be adjusted to accommodate different milling depths.

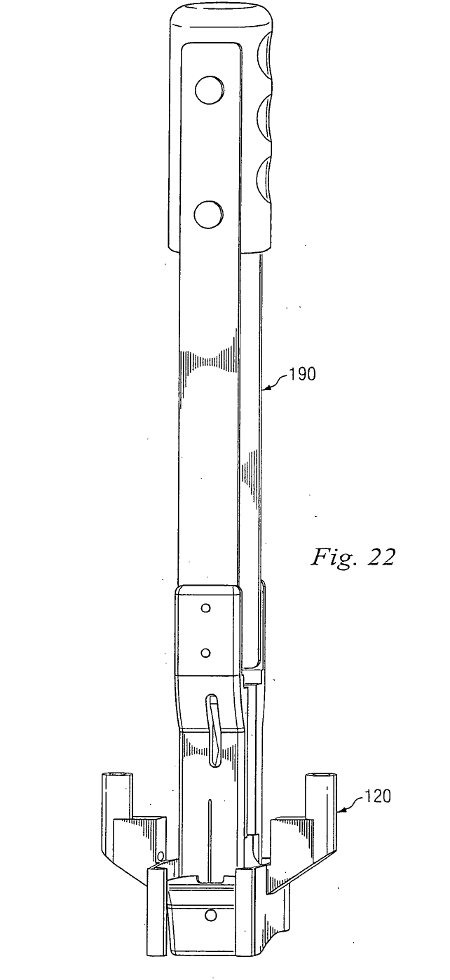

Referring now to FIG. 22, a set insertion prongs 290 may engage the prosthesis 18 and may pass the prosthesis through the milling fixture 220 to seat the prosthesis between the endplates of vertebral bodies 14, 16. To seat the prosthesis 18, the exterior surfaces and projections of the prosthesis 18 may engage the precision milled contours 244 of the vertebral bodies 14, 16. The precision matching of the milled contours 244 to the prosthesis 18 may provide anterior-posterior and transverse stability to the implanted prosthesis.

The implanted prosthesis 18 may permit translation along the anterior-posterior axis 49. In at least one embodiment, the translation may be approximately 3 millimeters. The implanted prosthesis 18 may also permit deflection in response to flexion-extension movement and lateral bending. In at least one embodiment, approximately 24 degrees of flexion-extension movement may be permitted. The contour 244 may be milled such that the implanted prosthesis 18 may be positioned in a flexion or extension position to permit a maximum range of spinal motion. The endplates of vertebrae 14, 16 may, for example, be milled to place the prosthesis in approximately 4 degrees of extension to bias the prosthesis 18 for flexion motion.

The embodiment as described above can be used as a prosthetic implant in a wide variety of joints, including hips, knees, shoulders, etc. The description below focuses on an embodiment wherein the implant is a spinal disc prosthesis, but similar principles apply to adapt the implant for use in other joints. Those of skill in the art will readily appreciate that the particulars of the internal geometry will likely require modification from the description below to prepare an implant for use in other joints. However, the concept of using a core body having geometric features adapted to interact with inner surfaces of opposing endplate assemblies to provide relatively unconstrained movement of the respective surfaces until the allowable range of motion has been reached, and the concept of encasing these surfaces in a fluid filled capsule formed by the opposing endplate assemblies and a flexible sheath, are applicable to use in any joint implant.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

What is claimed is:

1. A vertebral implant for interposition between two vertebral endplates, the implant comprising:
a first endplate assembly comprising a first restraint mechanism extending from a first exterior surface for engaging a first vertebral endplate;
a second endplate assembly comprising a second restraint mechanism extending from a second exterior surface for engaging a second vertebral endplate; and
a central body articulable between the first and second endplate assemblies,
wherein the first restraint mechanism has a shape that matches a contour in the first vertebral endplate.

2. The vertebral implant of claim 1 wherein the second restraint mechanism has a shape that matches a precision cut contour in the first vertebral endplate.

3. The vertebral implant of claim 1 wherein the contour is a precision cut contour in the first vertebral endplate, the precision cut contour formed by a rotating burr.

4. The vertebral implant of claim 1 wherein the shape of the first restraint mechanism is approximately D-shaped.

5. The vertebral implant of claim I wherein first restraint comprises a first restraint surface anteriorly facing and generally perpendicular to the first exterior surface and further comprises a second curvilinear restraint surface extending between the first restraint surface and the first exterior surface.

6. The vertebral implant of claim 5 wherein the first restraint surface is flat.

7. The vertebral implant of claim 1 wherein first restraint comprises a first restraint surface posteriorly facing and generally perpendicular to the first exterior surface and further comprises a second curvilinear restraint surface extending between the first restraint surface and the first exterior surface.

8. The vertebral implant of claim 1 wherein the first endplate assembly comprises a third restraint mechanism extending from the first exterior surface for engaging the first vertebral endplate.

9. The vertebral implant of claim I wherein the first restraint has a transverse dimension and an anterior-posterior dimension, wherein the transverse dimension is smaller than the anterior posterior dimension.

10. The vertebral implant of claim 1 wherein the first restraint comprises at least one aperture.

11. The vertebral implant of claim I wherein the precision cut contour positions the implant in an extension position.

12. The vertebral implant of claim 10 wherein the extension position is approximately a four degree extension position.

13. A method of implanting a vertebral implant, the implant comprising a central body articulable between first and second endplate assemblies, between two vertebral endplates, the method comprising:
positioning a rotable burr between a first vertebral endplate and a second vertebral endplate;
moving the burr in a transverse direction;
removing bone from a first vertebral endplate to form a first contour;
inserting the implant between the first and second endplate assemblies; and
positioning the first endplate assembly in contact with the first contour,
wherein the shape of the first endplate assembly matches the shape of the first contour.

14. The method of claim 13 further comprising controlling the transverse movement of the burr to a linear motion.

15. The method of claim 13 further comprising controlling the transverse movement of the burr to an arc-shaped motion.

16. The method of claim 13 further comprising inserting the implant through a milling fixture.

17. The method of claim 13 further comprising
removing bone from a second vertebral endplate to form a second contour;
positioning the second endplate assembly in contact with the second contour,
wherein the shape of the first endplate assembly matches the shape of the first contour.

18. The method of claim 17 wherein first and second contours are formed to position the vertebral implant in a deflected position.

19. The method of claim 18 wherein the vertebral implant positioned in the deflected position places the vertebral endplates in approximately 4 degrees of extension.

20. The method of claim 17 wherein a single burr creates the first and second contours simultaneously.

21. The method of claim 13 further comprising controlling a depth position of the rotable burr with a ratchet assembly.

22. The method of claim 13 further comprising controlling the movement of the burr in the transverse direction with a rack and pinion system.

23. The method of claim 13 further comprising controlling the movement of the burr in the transverse direction with a pivoting yoke system.

24. The method of claim 13 wherein the first contour comprises a first retention recess for retaining a first retention member of the first endplate assembly.

25. The method of claim 13 wherein the shape of the burr corresponds to the shape of the first endplate assembly.

26. A vertebral implant for interposition between two vertebral endplates, the implant comprising:
a first endplate assembly tapered toward a first posterior edge, the first endplate assembly comprising a first tab extending from a first exterior surface for engaging a first vertebral endplate;
a second endplate assembly tapered toward a second posterior edge, the second endplate assembly comprising a second tab extending from a second exterior surface for engaging a second vertebral endplate; and
a central body articulable between the first and second endplate assemblies,
wherein the first tab has a transverse dimension and an anterior-posterior dimension, and further wherein the transverse dimension is smaller than the anterior posterior dimension.

27. The vertebral implant of claim 26 wherein the first tab is a self-cutting keel comprising a tapered edge.

28. The vertebral implant of claim 26 wherein a posterior distance between the first tab and the first posterior edge is greater than an anterior distance between the first tab and an anterior edge of the first endplate assembly.

29. The vertebral implant of claim 26 wherein the first tab is formed from a polished metal.

30. The vertebral implant of claim 26 wherein the first endplate assembly further comprises an engagement element for mating with a revision tool.

31. The vertebral implant of claim 26 wherein the first tab has a proximal portion and a distal portion, and further wherein the distal portion is wider than the proximal portion.

32. The vertebral implant of claim 26 wherein the central body comprises a central anchoring recess engaged with a central anchoring post on the first endplate assembly to limit motion between the central body and the first endplate assembly.
